(19) United States
(12) Patent Application Publication
(10) Pub. No.: US 2026/0069620 A1
(43) Pub. Date: Mar. 12, 2026

(54) IMMUNOTHERAPEUTIC PHOSPHOLIPIDS FOR CANCER TREATMENT

(71) Applicant: University of Cincinnati, Cincinnati, OH (US)

(72) Inventor: Xiaoyang Qi, Blue Ash, OH (US)

(73) Assignee: University of Cincinnati, Cincinnati, OH (US)

(21) Appl. No.: 19/106,530

(22) PCT Filed: Aug. 29, 2023
(86) PCT No.: PCT/US2023/031350

§ 371 Date: Feb. 25, 2025

Related U.S. Application Data
(60) Provisional application No. 63/401,889, filed on Aug. 29, 2022.

(51) Int. Cl. A61K 31/683; A61K 9/00; A61K 9/127; A61P 35/00; A61P 37/04

(52) U.S. Cl. CPC A61K 31/683; A61K 9/0019; A61K 9/127; A61P 35/00; A61P 37/04

(57) ABSTRACT

A method of treating cancer in a subject is provided, the method including administering to the subject a therapeutic amount of a composition including phosphatidylglycerol nanovesicles (NVs). A method of inhibiting M2 macrophage polarization in a tumor microenvironment by administering phosphatidylglycerol NVs is also provided, together with pharmaceutical compositions including phosphatidylglycerol NVs.

### CROSS REFERENCE TO RELATED APPLICATION

This application claims priority to U.S. Provisional Application Ser. No. 63/401,889, filed Aug. 29, 2022, the entire contents of which are incorporated herein by reference.

### TECHNICAL FIELD

The present disclosure relates to the field of cancer treatment. Specifically, this disclosure relates to immunotherapeutic phospholipid nanovesicles for use in cancer treatment.

### BACKGROUND

Cancer cells have evolved mechanisms to establish an immunosuppressive tumor microenvironment that promotes escape from host immune attack and sustains tumor growth. The tumor microenvironment is a complex milieu comprised of many secreted factors and diverse cell types. During tumor progression, circulating monocytes and resident macrophages are recruited to the tumor site.

Macrophages polarize to either the M1 or M2 phenotypes in the tumor microenvironment. Classically activated M1 macrophages are typically considered anti-tumor, while alternatively activated M2 macrophages (tumor-associated macrophages, or TAMs) contribute to tumor survival through immune suppression, angiogenic/lymphangiogenic regulation, induction of hypoxia, tumor cell proliferation, and metastasis.

Inhibition of tumor macrophage M2 polarization is a compelling therapeutic approach for cancer treatment. A need exists for therapeutic compositions and methods that inhibit intra-tumor M2 macrophage polarization, thereby reducing the immunosuppressive nature of the tumor microenvironment to treat cancer.

### SUMMARY

It has now been discovered that cancer-secreted heat shock protein 70 (Hsp70) induces M2 macrophage polarization in the tumor microenvironment and that sequestration of Hsp70 by immunotherapeutic phospholipid nanovesicles reduces tumor growth. Accordingly, provided herein are compositions and methods that sequester cancer-secreted Hsp70 to inhibit M2 macrophage polarization, reduce tumor volume, and treat cancer.

In one embodiment, a method of treating cancer in a subject in need thereof is provided, the method comprising administering to the subject a therapeutic amount of a composition comprising phosphatidylglycerol nanovesicles (NVs).

In another embodiment, a method of inhibiting M2 macrophage polarization in a tumor microenvironment is provided, the method comprising administering to the tumor microenvironment an effective amount of a composition comprising phosphatidylglycerol NVs.

In another embodiment, a pharmaceutical composition for the treatment of cancer is provided, comprising: a therapeutic amount of phosphatidylglycerol NVs; and at least one pharmaceutically acceptable excipient, wherein the phosphatidylglycerol NVs do not encapsulate a second therapeutic agent and are not conjugated to a second therapeutic agent.

In another embodiment, a composition comprising phosphatidylglycerol nanovesicles (NVs) for use in a method of treating cancer is provided, the method comprising administering the composition to the subject.

These and other features, aspects, and advantages will become better understood with reference to the following description and the appended claims.

Additional features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

### BRIEF DESCRIPTION OF THE DRAWINGS

FIG. 1 is a graph showing results of flow cytometric analyses of THP-1 differentiation marker CD14 on THP-1 cells in response to Gli36 CM together with mAbs against alpha enolase, Hsp70, moesin, and S5A.

FIG. 2 is a graph showing results of flow cytometric analyses of THP-1 differentiation marker CD14 on THP-1 cells in response to increasing concentration of Hsp70 in Gli36 conditioned media (CM).

FIG. 3 is a graph showing results of data mined from The Cancer Genome Atlas Program (TCGA) database, showing higher HSP70A1A mRNA expression in glioma cancer compared to normal cells.

FIG. 4 is a graph showing results of data mined from the TCGA database, showing higher HSP70A1A mRNA expression in pancreatic cancer compared to normal cells.

FIG. 5 is a Western blot of whole cell lysates and the CMs obtained from the indicated cell lines probed with anti-Hsp70 mAb (top row) and anti-actin mAb (bottom row).

FIG. 6 shows Western blots analyses of whole cell lysates from WT (control shRNA), shHsp70 #1 or shHsp70 #2 from LLC-GFP cells (top left panel) and LN229 cells (top right panel) probed with anti-Hsp70 mAbs or anti-actin mAbs; and CD14 expression on THP-1 cells cultured with CM from WT (control shRNA) or shHsp70 #2 expressing LLC-GFP cells (bottom left panel) and LN229 cells (bottom right panel) measured by flow cytometry.

FIG. 7 shows a schematic for implantation of subcutaneous tumors from LLC-GFP cells in mice flank (top panel) and a graph of subcutaneous tumor volume from WT LLC-GFP cells (Line 1) or Hsp70 KD LLC-GFP cells (Line 2) over time (bottom panel).

FIG. 8 depicts flow cytometric measurement of M2 macrophage (MØ) marker CD206 expression in macrophages isolated from subcutaneous tumors from WT LLC-GFP cells expressing control shRNAs, and Hsp70 KD tumors (top left two panels and bottom left panel) and of M1 macrophage marker iNOS expression in macrophages isolated from subcutaneous tumors from WT LLC-GFP cells expressing control shRNAs, and Hsp70 KD tumors (top right two panels and bottom right panel.

FIG. 9 is a graph depicting CD14 expression measured by flow cytometry in THP-1 cells incubated MiaPaCa-2 CM with 100 μM of indicated phospholipid nanovesicles (NVs).

FIG. 10 is a graph depicting dose dependent inhibition of THP1 differentiation by DOPG NVs compared to DSPG NVs.

FIG. 11 depicts flow cytometric dot plots of propidium iodide positive cells upon incubation of THP-1 cells with MiaPaCa-2 CM and MiaPaCa-2 CM together with 100 μM of indicated PL NVs.

FIG. 12 depicts graphs showing quantification of Hsp70 by ELISA in pellets (top panel) and supernatants (bottom panel) obtained after 3 hrs incubation of Gli36 CM with indicated PL NVs and ultracentrifugation.

FIG. 13 is a Western blot detecting Hsp70 from supernatants and pellets obtained after 3 hrs incubation of Gli36 CM with indicated PL NVs and ultracentrifugation.

FIG. 14 depicts a schematic of implantation of LLC-GFP cells into mouse flank and DOPG NVs treatment regimen (top panel); and graphs showing body weights of mice (bottom left panel) and tumor volume in mice (bottom right panel) in response to PBS injection or DOPG NVs injection.

FIG. 15 is a graph showing intra tumor M2 polarized MOs polarized in PBS or DOPG NV-injected mice.

FIG. 16 is a graph of propidium iodide (PI) staining of NV-treated Mia-Pa-Ca-2 cells showing no cytotoxicity.

### DETAILED DESCRIPTION

The details of embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an” and “the” include plural references unless the content clearly dictates otherwise.

As used herein, the term “subject” generally refers to a living being (e.g., animal or human) capable of suffering from cancer. In a specific embodiment, the subject is a mammal, such as a human, rat, mouse, monkey, horse, cow, pig, dog, cat, guinea pig, etc. In a more specific embodiment, the subject is a human subject, a rat, or a mouse. In a more specific embodiment, the subject is a human.

The terms “treat,” “treatment,” and “treating,” as used herein, refer to a method of alleviating or abrogating a disease, disorder, and/or symptoms thereof. In a specific embodiment, the disease or disorder is cancer. In a very specific embodiment, the cancer is a type of cancer that secretes heat shock protein 70 (Hsp70), wherein the Hsp70 polarizes macrophages to the M2 phenotype in the tumor microenvironment.

As used herein, the terms “administer” or “administration” may comprise administration routes such as enteral (e.g., oral, sublingual, buccal, or rectal), parenteral (e.g., intravenous, intramuscular, subcutaneous, intraarterial, intrathecal, intratumoral, etc.), intranasal, inhaled, vaginal, transdermal, etc., so long as the route of administration results in treatment of cancer. In specific embodiments, the administration route is parenteral. In a very specific embodiment, the administration route is intravenous or intratumoral.

“Phosphatidylglycerol” or “PG,” as used herein, refers to a phospholipid having two acyl chains esterified to a glycerol, which in turn is bonded to a headgroup structure that contains one phosphate and no other groups with compensating positive charges (the remaining structure is a glycerol), such that the PG carries a net negative charge. PG is characterized by two chiral centers (the sn-2 position in the phosphatidyl group and the central carbon of the alcohol glycerol). In embodiments, PG lipids comprise a more unsaturated chain occupying the sn-1 position. An exemplary structure of PG is set forth below as Formula I.

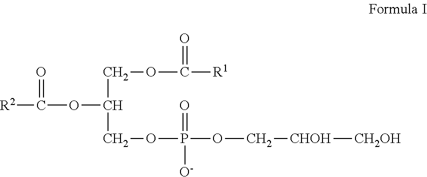

### General Chemical Structure of a Phosphatidylglycerol, where R¹ and R² are Fatty Acid Side Chains

In specific embodiments, phosphatidylglycerol lipids suitable for use in the methods and compositions disclosed herein include, but are not limited to, 1,2-dioleoyl-sn-glycero-3-phosphoglycerol (DOPG); 1,2-dioleoyl-sn-glycero-3-phospho-(1′-rac-glycerol) (18:1 (Δ9-Cis) PG); 1,2-dielaidoyl-sn-glycero-3-phospho-(1′-rac-glycerol) (18:1 (Δ9-Trans) PG); 1,2-dilinoleoyl-sn-glycero-3-phospho-(1′-rac-glycerol) (18:2 PG); 1,2-dilinolenoyl-sn-glycero-3-phospho-(1′-rac-glycerol) (18:3 PG); 1,2-diarachidonoyl-sn-glycero-3-[phospho-rac-(1-glycerol)](20:4 PG); 1,2-didocosahexaenoyl-sn-glycero-3-[phospho-rac-(1-glycerol)](22:6 PG); 1-palmitoyl-2-oleoyl-sn-glycero-3-phospho-(1′-rac-glycerol) (16:0-18:1 PG); 1-palmitoyl-2-linoleoyl-sn-glycero-3-phospho-(1′-rac-glycerol) (16:0-18:2 PG); 1-stearoyl-2-oleoyl-sn-glycero-3-phospho-(1′-rac-glycerol) (18:0-18:1 PG); 1-stearoyl-2-linoleoyl-sn-glycero-3-phospho-(1′-rac-glycerol) (18:0-18:2 PG); 1-(10Z-heptadecenoyl)-sn-glycero-3-phospho-(1′-rac-glycerol) (17:1 Lyso PG); 1-oleoyl-2-hydroxy-sn-glycero-3-phospho-(1′-rac-glycerol) (18:1 Lyso PG), and combinations thereof. In a very specific embodiment, the nanovesicles are DOPG nanovesicles. In embodiments, the nanovesicles.

“Nanovesicles” are generally spherical shaped lipid bilayer vesicles comprised of a lipid, such as a phospholipid. In embodiments, nanovesicles for use in the presently disclosed methods and compositions are phosphatidylglycerol nanovesicles. In embodiments, the nanovesicles have a diameter ranging from about 20 nm to about 200 nm.

“Co-administered,” as used herein, refers to administration of the disclosed phosphatidylglycerol NV compositions and a second therapeutic agent, such that both agents can simultaneously achieve a physiological effect, e.g., in a recipient subject. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the subject. Thus, in embodiments, the phosphatidylglycerol NV composition and the second therapeutic agent may be administered concurrently or sequentially.

“Effective amount,” as used herein, refers to an amount of an agent sufficient to achieve a desired biological effect. Effective amounts will vary based on a subject's age, body weight, condition, and the like, and may be determined by one of skill in the art in view of the present disclosure. The compositions of the present disclosure can be administered by either single or multiple dosages of an effective amount. In embodiments, the effective amount of an agent is an amount sufficient to treat cancer. In specific embodiments, the effective amount is an amount sufficient to sequester cancer-secreted Hsp70 in a tumor microenvironment to influence M2 macrophage polarization toward the M1 phenotype.

The development of strategies to block the generation of immunosuppressive M2 macrophages requires the identification of mechanisms by which cancer-derived factors generate M2 polarized macrophages and the therapeutic blockade of these molecules to disrupt the immunosuppressive communication between tumor cells and immune cells.

Heat shock protein 70 is one of many proteins implicated in the promotion of cancer cell growth. Although Hsp70 was originally discovered as an intracellular chaperone protein involved in the cellular stress response, Hsp70 is now known to be overexpressed in a variety of cancers. Depletion of Hsp70 has been shown to reduce tumor growth in pancreatic ductal adenocarcinoma, glioblastoma, colon, prostate, and hepatocellular carcinomas. Furthermore, both plasma membrane bound- and circulating-Hsp70 are increased in patients with glioblastoma, pancreatic cancer, and lung cancer. Hsp70 lacks a conventional secretory signal. Its secretion is thought to occur via a non-conventional mode involving lysosomal endosomes or by association with membrane rafts and other secretory proteins. Post-translational modifications of Hsp70 such as phosphorylation play critical roles in chaperone function. Hsp70 is thought to have increased phosphorylation at multiple sites in cancer cells due to increased kinase activity.

Unlike intracellular Hsp70, the function of secreted Hsp70 on tumor growth has not been well understood, in part because the extracellular receptor-mediated mechanisms of action are obscure. Although Hsp70 is known to bind the toll-like receptors (TLRs), a family of receptors well-documented for their pro-inflammatory function, the functional implications have been unclear. TLRs, expressed abundantly on macrophages, bind specifically to a broad spectrum of bacterial or pathogen structures triggering inflammatory responses. A role for macrophage TLRs in the regulation of tumor-induced immunosuppression is also not clear. The Tyro3, Axl, and Mer receptor tyrosine kinases play a crucial role in macrophage M2 polarization in the tumor microenvironment, but it has not been determined whether TLRs communicate with these receptors in the tumor microenvironment to exert macrophage M2 polarization.

The present investigators have now discovered that the predominant macrophage polarizing activity in conditioned media (CM) can be accounted for by secreted Hsp70. Cellular Hsp70 knockdown led to decreased tumor growth and reduction in intra-tumor M2 polarized macrophages. Infusion of PG NVs that bind secreted Hsp70 inhibited cancer-induced macrophage differentiation toward the M2 phenotype, intra-tumor M2 macrophages, and tumor growth in mice.

### Methods of Use

In one embodiment, a method of treating cancer in a subject in need thereof is provided, the method comprising administering to the subject a therapeutic amount of a composition comprising phosphatidylglycerol nanovesicles (NVs). In embodiments, the phosphatidylglycerol NVs sequester secreted (Hsp70, and specifically cancer-secreted Hsp70 in the tumor microenvironment (TME) and/or circulating secreted Hsp70.

While not desiring to be bound by theory, it is believed that the extent of unsaturation and the area per lipid (APL) of the phosphatidylglycerol, in combination with the net negative charge of the phosphatidylglycerol, are factors that contribute to the successful scavenging of secreted Hsp70 by phosphatidylglycerol NVs. In embodiments, the phosphatidylglycerol is unsaturated. In specific embodiments, the phosphatidylglycerol has an area per lipid (APL) greater than about 70 Å².

In embodiments, the composition comprising phosphatidylglycerol NVs does not comprise a second therapeutic agent. In embodiments, the phosphatidylglycerol NVs are not conjugated to and/or do not encapsulate a second therapeutic agent. That is, in embodiments, the phosphatidylglycerol NVs are the sole therapeutic agent in the composition for use in treating cancer. In embodiments, the composition consists essentially of phosphatidylglycerol NVs. In a very specific embodiment, the phosphatidylglycerol is DOPG.

In embodiments, the subject of the methods provided herein is a mammal, such as a human, rat, mouse, monkey, horse, cow, pig, dog, cat, guinea pig, etc. In a more specific embodiment, the subject is a human subject, a rat, or a mouse. In a more specific embodiment, the subject is a human.

In embodiments, the route of administration of the compositions comprising phosphatidylglycerol NVs may be enteral (e.g., oral, sublingual, buccal, or rectal), parenteral injection or infusion (e.g., intravenous, intramuscular, subcutaneous, intraarterial, intrathecal, intratumoral, etc.), intranasal, inhaled, vaginal, transdermal, etc., so long as the route of administration results in treatment of cancer and/or sequestration of secreted Hsp70 in a tumor microenvironment. In specific embodiments, the administration route is parenteral. In a more specific embodiment, the administration route is via injection or infusion.

Suitable cancers for treatment by the presently disclosed methods include those cancers characterized by secretion and/or elevated secretion of Hsp70 in a tumor microenvironment. In embodiments, such cancers produce tumors, and more specifically solid tumors. In specific embodiments, the cancer includes, but is not limited to, pancreatic ductal adenocarcinoma, glioblastoma, colorectal cancer, prostate cancer, hepatocellular carcinoma, lung cancer, skin cancer, cervical cancer, ovarian cancer, endometrial cancer, myeloma, acute myeloid leukemia, chronic lymphocytic leukemia, and diffuse large-B cell lymphoma. It will be appreciated that other forms of cancer may also be treated by the methods and compositions disclosed herein.

In another embodiment, a method of inhibiting M2 macrophage polarization in a tumor microenvironment is provided, the method comprising administering to the tumor microenvironment an effective amount of a composition comprising phosphatidylglycerol nanovesicles (NVs). In embodiments, the phosphatidylglycerol is unsaturated. In further embodiments, the phosphatidylglycerol has an area per lipid (APL) greater than about 70 Å².

In embodiments, the method of inhibiting M2 macrophage polarization may be carried out in vitro or in vivo. In embodiments, the phosphatidylglycerol NVs sequester secreted Hsp70, and specifically circulating cancer-secreted Hsp70 and/or cancer-secreted Hsp70 in the tumor microenvironment. Advantageously, sequestering secreted Hsp70 in the tumor microenvironment shifts macrophage polarization primarily to the M1 phenotype, thereby reducing the immunosuppressive nature of the tumor microenvironment. In embodiments, the methods disclosed herein sequester secreted Hsp70, which leads to a reduction in tumor volume and/or inhibition of tumor growth.

In embodiments, the composition comprising phosphatidylglycerol NVs does not comprise a second therapeutic agent. In embodiments, the phosphatidylglycerol NVs are not conjugated to and/or do not encapsulate a second therapeutic agent. That is, in embodiments, the phosphatidylglycerol NVs are the sole therapeutic agent in the composition for use in inhibiting macrophage M2 polarization. In embodiments, the composition consists of or consists essentially of phosphatidylglycerol NVs. In a very specific embodiment, the composition consists of or consists essentially of DOPG NVs.

In embodiments, the tumor microenvironment is associated with a cancer including, but not limited to, pancreatic ductal adenocarcinoma, glioblastoma, colorectal cancer, prostate cancer, hepatocellular carcinoma, lung cancer, skin cancer, cervical cancer, ovarian cancer, endometrial cancer, myeloma, acute myeloid leukemia, chronic lymphocytic leukemia, and diffuse large-B cell lymphoma. In specific embodiments, the tumor is a solid tumor.

In embodiments, a second therapeutic agent may be co-administered with the composition comprising phosphatidylglycerol NVs. In embodiments, the phosphatidylglycerol NV composition and the second therapeutic agent may be administered concurrently or sequentially.

With regard to sequential administration, the phosphatidylglycerol NVs and the second therapeutic agent may be administered within one hour, within two hours, within four hours, within 8 hours, within 24 hours, within two days, within three days, within four days, within five days, within six days, or within one week of each other. In embodiments, phosphatidylglycerol NVs are administered first, followed by the second therapeutic agent. In embodiments, the second therapeutic agent is administered first, followed by phosphatidylglycerol NVs.

Suitable therapeutic agents that may be co-administered with the phosphatidylglycerol NVs of the present disclosure include any therapeutic agent that may be administered to a patient undergoing cancer treatment. Exemplary therapeutic agents are set forth in U.S. Pat. No. 10,787,440, issued Sep. 29, 2020 to Keilhack, et al., which is incorporated herein by reference in its entirety. The therapeutic agents set forth below are for illustrative purposes and not intended to be limiting. It will be appreciated that any therapeutic agent appropriate for treatment of a particular cancer at issue may be selected for co-administration with a composition comprising phosphatidylglycerol NVs.

In embodiments, the second therapeutic agent is an anticancer agent. In one embodiment, the anticancer agent is a compound that affects histone modifications, such as an HDAC inhibitor. In certain embodiments, an anticancer agent is selected from the group consisting of chemotherapeutics (such as 2CdA, 5-FU, 6-Mercaptopurine, 6-TG, Abraxane™ Accutane®, Actinomycin-D, Adriamycin®, Alimta®, all-trans retinoic acid, amethopterin, Ara-C, Azacitadine, BCNU, Blenoxane®, Camptosar®, CeeNU®, Clofarabine, Clolar™, Cytoxan®, daunorubicin hydrochloride, DaunoXome®, Dacogen®, DIC, Doxil®, Ellence®, Eloxatin®, Emcyt®, etoposide phosphate, Fludara®, FUDR®, Gemzar®, Gleevec®, hexamethylmelamine, Hycamtin®, Hydrea®, Idamycin®, Ifex®, ixabepilone, Ixempra®, L-asparaginase, Leukeran®, liposomal Ara-C, L-PAM, Lysodren, Matulane®, mithracin, Mitomycin-C, Myleran®, Navelbine®, Neutrexin®, nilotinib, Nipent®, Nitrogen Mustard, Novantrone®, Oncaspar®, Panretin®, Paraplatin®, Platinol®, prolifeprospan 20 with carmustine implant, Sandostatin®, Targretin®, Tasigna®, Taxotere®, Temodar®, TESPA, Trisenox®, Valstar®, Velban®, Vidaza™, vincristine sulfate, VM 26, Xeloda® and Zanosar®); biologies (such as Alpha Interferon, Bacillus Calmette-Guerin, Bexxar®, Campath®, Ergamisol®, Erlotinib, Herceptin®, Interleukin-2, Iressa®, lenalidomide, Mylotarg®, Ontak®, Pegasys®, Revlimid®, Rituxan®, Tarceva™, Thalomid®, Tykerb®, Velcade® and Zevalin™); corticosteroids, (such as dexamethasone sodium phosphate, DeltaSone® and Delta-Cortef®); hormonal therapies (such as Arimidex®, Aromasin®, Casodex®, Cytadren®, Eligard®, Eulexin®, Evista®, Faslodex®, Femara®, Halotestin®, Megace®, Nilandron®, Nolvadex®, Plenaxis™ and Zoladex®); and radiopharmaceuticals (such as Iodotope®, Metastron®, Phosphocol® and Samarium SM-153).

In another embodiment, the second therapeutic agent is a chemotherapeutic agent (also referred to as an anti-neoplastic agent or anti-proliferative agent), selected from the group including an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue drug, or any chemotherapeutic, anti-neoplastic or anti-proliferative agent.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) and mesna (Mesnex).

Exemplary interferons include, but are not limited to, interferon alfa-2b (Intron A) and interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/iodinel31 tositumomab (Bexxar); alemtuzumab (Campath); ibritumomab (Zevalin; In-111; Y-90 Zevalin); gemtuzumab (Mylotarg); and eculizumab (Soliris) ordenosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) and EKB-569.

Exemplary HER2 inhibitors include, but are not limited to, trastuzumab (Herceptin); lapatinib (Tykerb) and AC-480.

Histone Deacetylase Inhibitors include, but are not limited to, vorinostat (Zolinza).

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); and testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); and amsacrine or lamellarin D (LAM-D).

Exemplary MTOR inhibitors include, but are not limited to, everolimus (Afinitor) or temsirolimus (Torisel); rapamune, ridaforolimus; and AP23573.

Exemplary VEGF/VEGFR inhibitors include, but are not limited to, bevacizumab (Avastin); sorafenib (Nexavar); sunitinib (Sutent); ranibizumab; pegaptanib; and vandetinib.

Exemplary microtubule targeting drugs include, but are not limited to, paclitaxel, docetaxel, vincristine, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur-0.4 M 5-chloro-2,4-dihydroxypyrimidine-1 M potassium oxonate), and lovastatin.

In another aspect, the second therapeutic agent is a chemotherapeutic agent or a cytokine such as G-CSF (granulocyte colony stimulating factor).

In another aspect, the second therapeutic agents can be standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar™), CHOP (cyclophosphamide, hydroxydaunorubicin, oncovin, and prednisone or prednisolone), R-CHOP (rituximab, cyclophosphamide, hydroxydaunorubicin, oncovin, prednisone or prednisolone), and CMFP (cyclophosphamide, methotrexate, 5-fluorouracil and prednisone).

In another aspect, the second therapeutic agents can be an inhibitor of an enzyme, such as a receptor or non-receptor kinase. Receptor and non-receptor kinases are, for example, tyrosine kinases or serine/threonine kinases. Kinase inhibitors described herein are small molecules, polynucleic acids, polypeptides, or antibodies.

Exemplary kinase inhibitors include, but are not limited to, Bevacizumab (targets VEGF), BIBW 2992 (targets EGFR and Erb2), Cetuximab/Erbitux (targets Erb 1), Imatinib/Gleevic (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb 1), Nilotinib (targets Bcr-Abl), Lapatinib (targets Erb 1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P 154 (targets JAK), WHI-P 131 (targets JAK), sorafenib/Nexavar (targets RAF kinase, VEGFR-1, VEGFR-2, VEGFR-3, PDGFR-β, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF 1-3, PDGFR, and c-kit), Denosumab (targets RANKL, inhibits SRC), AMG888 (targets HER3), and AP24534 (multiple targets including Flt3).

Exemplary serine/threonine kinase inhibitors include, but are not limited to, Rapamune (targets mTOR/FRAP1), Deforolimus (targets mTOR), Certican/Everolimus (targets mTOR/FRAP1), AP23573 (targets mTOR/FRAP1), Eril/Fasudil hydrochloride (targets RHO), Flavopiridol (targets CDK), Seliciclib/CYC202/Roscovitrine (targets CDK), SNS-032/BMS-387032 (targets CDK), Ruboxistaurin (targets PKC), Pkc412 (targets PKC), Bryostatin (targets PKC), KAI-9803 (targets PKC), SF 1126 (targets P13K), VX-680 (targets Aurora kinase), Azdl 152 (targets Aurora kinase), Arry-142886/AZD-6244 (targets MAP/MEK), SCIO-469 (targets MAP/MEK), GW681323 (targets MAP/MEK), CC-401 (targets JNK), CEP-1347 (targets JNK), and PD 332991 (targets CDK).

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; and AMG888.

### Pharmaceutical Compositions

In another embodiment, a pharmaceutical composition for the treatment of cancer is provided, the pharmaceutical composition comprising: a therapeutic amount of phosphatidylglycerol nanovesicles (NVs); and at least one pharmaceutically acceptable excipient. In embodiments, the phosphatidylglycerol NVs do not encapsulate a second therapeutic agent and are not conjugated to a second therapeutic agent. In a specific embodiment, the phosphatidylglycerol NVs are the sole therapeutic agent in the pharmaceutical composition.

In embodiments, the phosphatidylglycerol is unsaturated. In further embodiments, the phosphatidylglycerol has an area per lipid (APL) greater than about 70 Å².

Phosphatidylglycerol NVs can be prepared according to methods known in the field. In embodiments, a desired amount of phosphatidylglycerol phospholipid is dissolved in an organic solvent and the solvent is removed by drying, e.g., drying using N₂ gas. The dried phospholipid film is then mixed with a pharmaceutically-acceptable carrier, such as PBS, and sonicated in a sonicator to generate phosphatidylglycerol nanovesicles as described herein.

In embodiments, pharmaceutical compositions comprising phosphatidylglycerol NVs may be formulated for enteral, parenteral, intranasal, inhaled, vaginal, transdermal, or other form of administration. In a specific embodiment, the pharmaceutical composition is formulated for parenteral administration. In a more specific embodiment, the pharmaceutical composition is formulated for intravenous administration via injection or infusion. In another specific embodiment, the pharmaceutical composition is formulated for intratumoral injection.

The compositions may be prepared by any methods well known in the art of pharmacy, for example, using methods such as those described in Remington: The Science and Practice of Pharmacy (23rd ed., Adeboye Adejare, ed., 2020, see Section 7: Pharmaceutical Materials and Devices/Industrial Pharmacy). Suitable pharmaceutical carriers are well-known in the art. See, for example, Handbook of Pharmaceutical Excipients, Sixth Edition, edited by Raymond C. Rowe (2009). The skilled artisan will appreciate that certain carriers may be more desirable or suitable for certain modes of administration of an active ingredient. It is within the purview of the skilled artisan to select the appropriate carriers for a given pharmaceutical composition.

For parenteral administration, suitable compositions include aqueous and non-aqueous sterile suspensions for intravenous administration. The compositions may be presented in unit dose or multi-dose containers, for example, sealed vials and ampoules.

In embodiments, the dosage or concentration of phosphatidylglycerol NVs in the compositions disclosed herein may range from 1 μM to 500 μM; from 25 μM to 500 μM; from 25 μM to 400 μM; from 25 μM to 350 μM; from 25 μM to 300 μM; from 25 μM to 250 μM; from 25 μM to 200 μM; from 25 μM to 150 μM; from 25 μM to 100 μM; from 50 μM to 350 μM; from 50 μM to 300 μM; from 50 μM to 250 μM; from 50 μM to 200 μM; from 50 μM to 150 μM; from 50 μM to 125 μM; from 50 μM to 100 μM; from 50 μM to 75 μM; from 100 μM to 350 μM; from 100 μM to 300 μM; from 100 μM to 250 μM; from 100 μM to 200 μM; from 100 μM to 150 μM; from 150 μM to 350 μM; from 150 μM to 300 μM; from 150 μM to 250 μM; from 150 μM to 200 μM; from 200 μM to 350; from 200 μM to 300 μM; from 200 μM to 250 μM; from 250 μM to 350 μM; from 250 μM to 300 μM; or any range therebetween.

As will be understood by those of skill in this art, the specific dose level for any particular subject will depend on a variety of factors, including the activity of the agent employed; the age, body weight, general health, and sex of the individual being treated; the time and route of administration; the rate of excretion; and the like.

### EXAMPLES

The following examples are given by way of illustration are not intended to limit the scope of the disclosure.

### Example 1. Materials and Methods

Cell Lines. The cell lines studied were human pancreatic cancer cell line (MiaPaCa-2), lung cancer cell lines (H1299, LLC-GFP), glioblastoma cell line (Gli36), human monocyte cell line (THP-1), human macrophage cell line (SC), and mouse macrophage cell line (J774), all obtained from ATCC.

Cell Culture. WT/TLR2 null THP-1 cells, human SC cells and J774 macrophage cells were cultured in RPMI with 25 mM HEPES. All other cell lines were cultured in DMEM and all media were supplemented with 10% FBS and 1% penicillin/streptomycin. All cells were cultured in a 5% CO₂ incubator at 37° C. Cells were routinely tested for mycoplasma contamination. No cross-contamination was observed in the cell lines, as determined by cellular morphology and growth parameters.

Generation of serum-free, exosome/microparticle-free CMs from human/mouse cancer cell lines and THP-1 differentiation assay. Human/mouse cancer cell lines were grown in their respective media until 70% confluency in 10 cm Corning tissue culture plates (ThermoFisher, MA), at which time the media was removed. The cells were washed twice with serum-free media to remove remnants of serum and dead cells and replenished with serum-free media. After 24 hrs, CM was collected, centrifuged at 10,000 g to remove cellular debris, followed by ultracentrifugation at 100,000 g to remove extracellular exosomes and microparticles. THP-1 cells (2×10⁵) were cultured in 1 ml of CM normalized to 100 μg of total cellular protein from indicated cancer cell lines. Control THP-1 cells were grown in DMEM. After 24 hrs, control and CM-treated cells were centrifuged then incubated with CD14-PE conjugated antibody (eBioscience, CA) and propidium iodide (PI; BD Biosciences, NJ) in 100 μl FACS buffer for 30 mins on ice. Cells were washed with flow cytometry buffer (PBS with 2% FBS) and CD14 expression measured by flow cytometry. In studies involving assessment of exosomes/microparticles in THP-1 differentiation, THP-1 cells were cultured in unfractionated CM, CM devoid of exosomes/microparticles, or with the exosomes/microparticles fraction of the CM for 24 hrs and differentiation was measured by flow cytometric assessment of CD14 expression as described above.

Flow cytometry. Cells were washed once with PBS, centrifuged and cell pellets were incubated on ice with respective antibodies in flow cytometry buffer (PBS with 2% FBS). After a 45 mins incubation, cells were washed twice with flow cytometry buffer and examined. THP-1 differentiation was assessed by flow cytometric measurement of PE-conjugated CD14 (eBioscience, CA). Propidium iodide (PI) was added to all stained cells to gate out dead cells. Stained cells were analyzed with a BD Fortessa flow cytometer.

Intracellular staining of cells for flow cytometry analyses. Cells were washed twice with PBS and fixed using 2% formaldehyde for 30 mins. After fixation, cells were spun down and washed two times with PBS and pelleted. Cell pellets were incubated with permeabilization buffer (eBioscience, CA) for 30 mins, followed by centrifugation. These cell pellets were incubated for 1 hr with antibodies against intracellular antigens in permeabilization buffer. After two washes with permeabilization buffer and one with flow cytometry buffer the cells were analyzed.

Lentiviral mediated KD of Hsp70. The pLKO.1 vectors expressing shRNAs targeting Hsp70 were obtained from Sigma-Aldrich, MO. The pLKO.1 lentiviruses were packaged in HEK-293T cells by co-transfecting the pMD2.G (VSV G) envelope plasmid and the Gag, Pol expressing psPAX2 packaging plasmid. These cells were cultured for 48 hrs after transfection and the lentiviral particles were collected from the supernatants and used to transduce LLC-GFP and LN229 cells. Gene silencing efficiency was analyzed by immunoblotting for Hsp70 at 36 hrs post-infection.

Quantification of Hsp70 in cancer cell CMs by ELISA. Human/mouse cancer cell lines were grown in their respective media until 70% confluency in 10 cm tissue culture plates (ThermoFisher, MA). After removing the media, the cells were washed twice with serum-free media to remove remnants of serum and dead cells and replenished with serum-free media. After 24 hrs, CM was collected by ultracentrifugation at 100,000 g to remove extracellular exosomes and microparticles. Hsp70 in the CM was quantified using an Hsp70 ELISA Kit (ThermoFisher, MA) according to the manufacturer's protocol.

Preparation of Phospholipid (PL) NVs and treatment of THP-1 cells. 1 M stock solution of PL NVs were prepared by taking desired amounts of individual PLs (DOPG, DOPA, DOPS, DSPC, or DSPG; Avanti Polar Lipids, AL) in organic solvent chloroform into glass tubes. The organic solvent was removed by drying using N₂ gas. To the dried film of lipids, 1 ml PBS was added and bath sonicated in ice-water bath sonicator for 30 mins to generate PL NVs. THP-1 cells were treated for 24 hrs with indicated cancer cell CMs with 50 to 100 μM of indicated PL NVs.

Affinity purification of Hsp70 and assessment of THP-1 differentiation activity. Gli36 CM (10, 20, and 30 ml) was concentrated to 1 ml, incubated with anti-Hsp70 specific mAb overnight at 4° C., followed by addition of protein AG beads then again incubated overnight at 4° C. Next, the beads were pelleted by centrifugation and washed 2 times with PBS. Bound proteins were eluted by using Tris (20 mM)-glycine (200 mM) buffers (pH 2.5, 3.0, 3.5, or 4.0). Eluates were neutralized by addition of an equal volume of pH 8.5 buffer. Alternatively, the beads were eluted with 3.5 MgCl₂ and the eluates were renatured by dialysis against PBS using a 10 kDa cut off dialysis membrane. In addition, proteins were removed from the antibodies using 8 M urea and the eluates were serially dialyzed against buffers containing 6, 4, and 2 M urea and finally against PBS. Eluates 100 μl together with 1 ml DMEM medium were added to THP-1 cells and after 24 hrs THP-1 differentiation was measured by CD14 expression analyses by flow cytometry.

Subcutaneous cancer cell implantation and DOPG NV treatment. WT and Hsp70 KD Lewis lung carcinoma cells (1×10⁵) expressing green fluorescent protein (LLC-GFP) were implanted subcutaneously into 6-8 weeks old C57BL/6J mice (5 mice/group). Treatment group (DOPG NVs in 200 μl PBS, 1.6 mg/kg per mouse) and control group (200 μl PBS/mouse) were conducted through tail vein injection. The treatments were started 2 days after implantation and continued thrice a week until the end of the treatments. Tumor growth was assessed daily by measuring the tumor volume. The tumors were collected when the volume reached 500 mm³ and used for histology and tumor macrophage analyses. Tumor volumes were measured by vernier calipers and calculated by using the formula V=(π/6)LW² (V, volume; L, length; W, width).

Tumor dissociation into single cells and flow cytometry. Freshly excised tumors were cut into small pieces and minced using a scalpel. The minced tumor tissue was incubated with 100 units of collagenase type 4 (Worthington Biochemicals, NJ) for 45 mins at 37° C. in RPMI medium. The collagenase treated tumor tissue was passed through 40 μm cell strainer (ThermoFisher, MA). The isolated tumor cells were washed twice with PBS and 5×10⁵ cells were incubated in 100 μl flow cytometry buffer (PBS with 2% FBS) with mouse Fc block for 30 mins on ice, followed by incubation with anti-mouse F4/80-PE (eBioscience, CA) and anti-mouse CD206 APC (BioLegend, CA) for 30 mins on ice. Cells were washed and fixed with fixation buffer (eBioscience, CA) for 30 mins. Fixed cells were centrifuged and washed twice with PBS. Cells were permeabilized using permeabilization buffer (eBioscience, CA) according to the manufacturer's protocol and then incubated with anti-mouse iNOS antibody (eBioscience, CA) in permeabilization buffer for 45 mins. Cells were washed twice with permeabilization buffer and finally washed with flow cytometry buffer. Macrophages were analyzed for M2 CD206+ cells and M1 iNOS+ cells by gating on F4/80 positive cells.

Cell sorting. Indicated cancer cell lines were stained with annexin V-FITC (Invitrogen, CA) and PI, according to the manufacturer's protocol. Briefly, 1×106 cells were incubated with annexin V binding buffer (Invitrogen, CA) together with PI for 30 mins at room temperature. Cells were washed with annexin V buffer, resuspended in this and cells with low annexin V signal and high annexin V signal were gated and sorted by flow cytometry.

SDS-page and western blotting analyses. For SDS-PAGE, 50 μg of whole cell lysates in RIPA buffer (Sigma-Aldrich, MO) were denatured in SDS-loading dye (Bio-Rad, CA) then loaded onto 4-15% denaturing gradient gels (Bio-Rad, CA). Proteins from the gel were transferred onto nitrocellulose membranes and blots were blocked with 5% non-fat dry milk in PBS-0.1% Tween-20, followed by the addition of protein specific antibodies and incubated overnight at 4° C. The blots were washed with PBS-Tween-20 three times and further incubated with HRP-coupled secondary antibodies. Following three washes with PBS-Tween-20, the blots were developed with SuperSignal West Dura (ThermoFisher, MA). For immunoprecipitates, the protein A+G agarose beads were boiled in SDS loading dye and loaded onto a gel and western blotting was performed.

Lipid binding assay. PL NVs were prepared as described above except that Hepes buffered saline was used instead of PBS. Gli36 CM was incubated with 100 μM of PL NVs for 3 hrs rotating at room temperature, ultracentrifuged at 170,000 g for 1 hr. Supernatant fractions were used for ELISA assays. Both pellet and supernatant fractions were used for western blot analyses.

Statistical analyses. GraphPad Prism 6 software was used for all statistical analyses. Data are presented as mean±SEM and were analyzed using a t-test (paired, non-parametric) for two group comparisons. Details of statistical analyses are provided in the figure legends. In vitro experiments were performed two to three times with consonant results. In vivo experiments were performed at least three times with consistent results. P values less than 0.05 were considered statistically significant.

### Example 2. Cancer-Secreted Hsp70 Stimulates THP-1 Cell Differentiation in a Dose Dependent Manner

The ability of monoclonal antibodies (mAbs) targeting alpha enolase, Hsp70, moesin, and S5A to inhibit THP-1 differentiation in response to Gli36 CM was assessed. Flow cytometric analysis of THP-1 differentiation marker CD14 was carried out on THP-1 cells in response to Gli36 CM together with indicated mAbs against indicated target proteins. 200 ng of the mAbs were used in THP-1 cell cultures in the presence of Gli36 CM equivalent to 7.4 ng Hsp70.

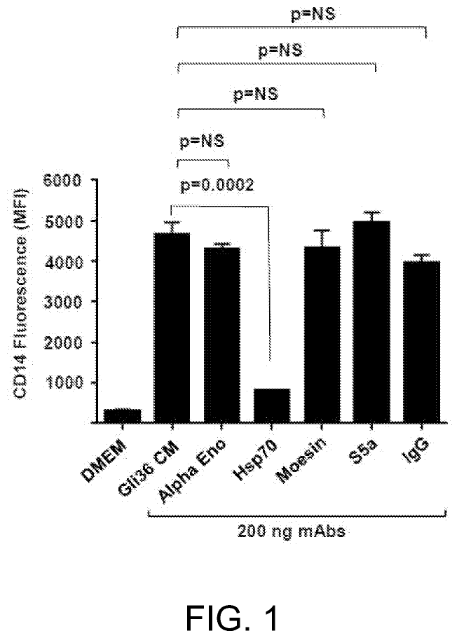

Monoclonal antibodies against Hsp70 showed the most potent inhibition of Gli36 CM-induced THP-1 differentiation, strongly suggesting that cancer-secreted Hsp70 stimulates THP-1 differentiation (FIG. 1).

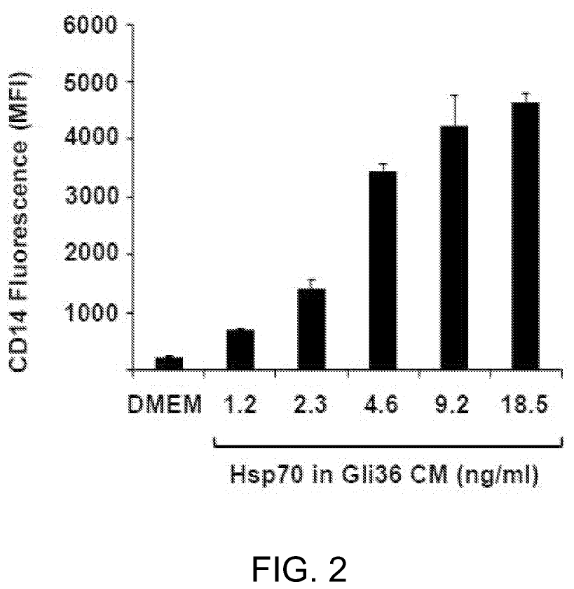

Flow cytometric analysis of the THP-1 differentiation marker CD14 was then carried out on THP-1 cells in response to increasing concentration of Hsp70 in Gli36 CM. Results showed Hsp70 concentration-dependent THP-1 differentiation activity of Gli36 CM (FIG. 2).

### Example 3. Hsp70 is Expressed in Glioma and Pancreatic Cancer

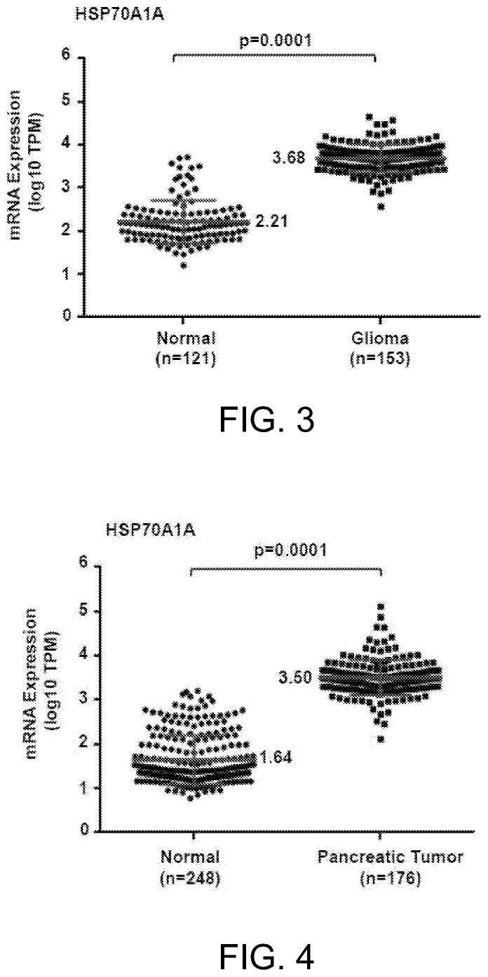

The Cancer Genome Atlas Program data was queried to assess Hsp70 mRNA expression in glioma (n=153) and pancreatic (n=176) cancer cells compared to normal cells (n=121 and n=248, respectively). Results, as shown in FIG. 3 (glioma) and FIG. 4 (pancreatic cancer), indicate that HSP70A1A mRNA expression is relatively higher in glioma and pancreatic cancer cells compared to their respective normal tissue cells.

### Example 4. Cancer-Secreted Hsp70 is a Mediator of THP-1 Differentiation

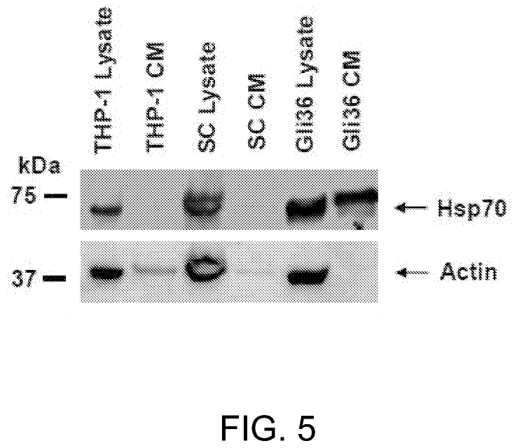

To rule out THP-1 and SC as a source of Hsp70, western blots were performed on lysates and conditioned media (CM) from these cells cultured in isolation. Hsp70 was not detected in the CMs of THP-1 and SC cells, compared to Gli36 CM (FIG. 5). These results indicate that the observed THP-1 differentiation is attributable to cancer-secreted Hsp70 in the CMs.

### Example 5. Hsp70 Knockdown in Cancer Cells Shifts Macrophage Differentiation to the M1 Phenotype

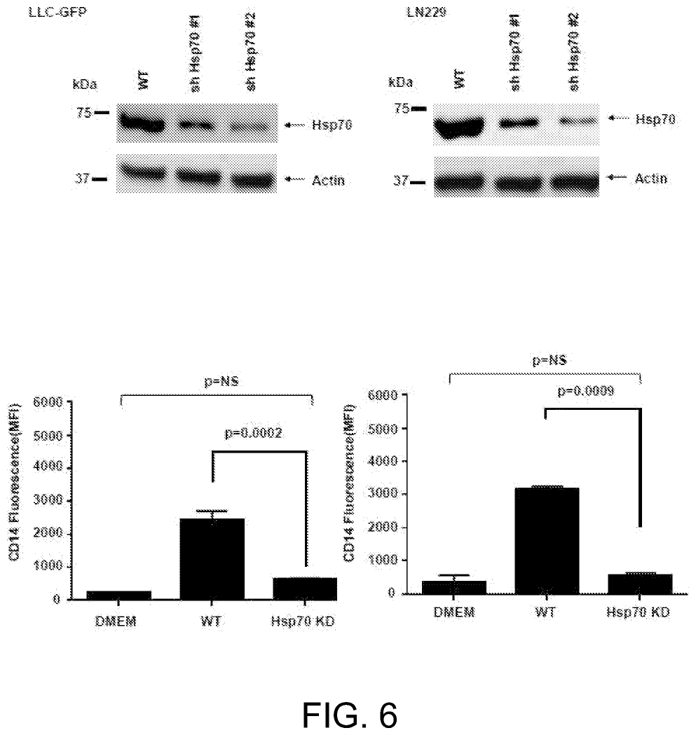

The influence of CM-derived Hsp70 on macrophage differentiation was studied with respect to tumor growth and intra-tumor macrophage polarization. Hsp70 knockdown (KD) was carried out by lentiviral-mediated expression of either control shRNAs (WT) or Hsp70 targeting shRNAs in two cancer cell lines, LLC-GFP and LN229. Western blots analyses of whole cell lysates from WT (control shRNA), shHsp70 #1 or shHsp70 #2 from LLC-GFP cells (FIG. 6, top left panel) and LN229 cells (FIG. 6, top right panel) probed with anti-Hsp70 mAbs or anti-actin mAbs are set forth. CD14 expression on THP-1 cells cultured with CM from WT (control shRNA) or shHsp70 #2 expressing LLC-GFP cells (FIG. 6, bottom left panel) and LN229 cells (FIG. 6, bottom right panel) were measured by flow cytometry.

In vitro, Hsp70 KD LLC-GFP and LN229 cells are viable and grew normally, comparable to WT cells, most likely because Hsp70 expression in not completely eliminated (FIG. 6, top left and right panels). KD of Hsp70 in LLC-GFP and LN229 cells led to a marked decrease in macrophage differentiation activity of the CMs obtained from LLC-GFP and LN229 cells compared to the CMs obtained from control shRNA expressing cells (FIG. 6, bottom left and right panels).

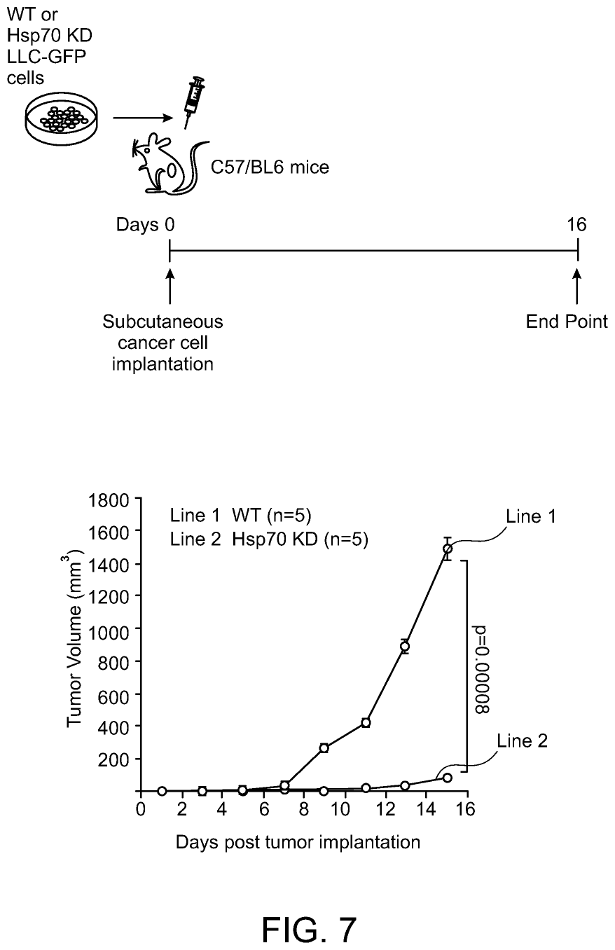

LLC-GFP cells were then implanted subcutaneously in the mouse flank (FIG. 7, top panel) and observed for 16 days. Tumor growth from LLC-GFP cells with Hsp70 KD (FIG. 7, bottom panel, Line 2) was severely impaired compared to LLC-GFP cells with control shRNAs (FIG. 7, bottom panel, Line 1).

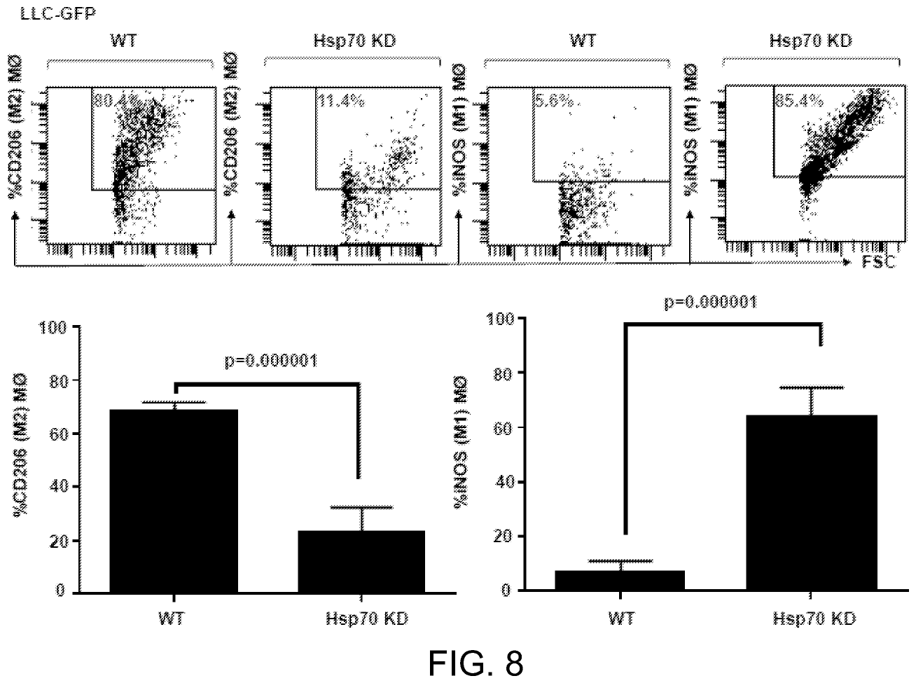

Flow cytometric measurement of M2 macrophage marker CD206 expression was carried out in macrophages isolated from subcutaneous tumors from WT LLC-GFP cells expressing control shRNAs and Hsp70 KD tumors (FIG. 8, top left two panels). Flow cytometric measurement of M1 macrophage marker iNOS expression was carried out in macrophages isolated from subcutaneous tumors from WT LLC-GFP cells expressing control shRNAs and Hsp70 KD tumors (FIG. 8, top right two panels).

Results showed that tumors from LLC-GFP cells with control shRNAs (WT) contained predominantly pro-tumorigenic M2 polarized macrophages (MØ), while tumors from Hsp70 KD cells were very small and contained M1 polarized macrophages. WT tumors had low M1 macrophages compared to Hsp70 KD tumors (FIG. 8, bottom left and right panels).

### Example 6. DOPG NVs Block Cancer Cell CM-Induced Monocyte Differentiation and Inhibit Tumor Growth in Mice

As studies indicated that cancer-secreted Hsp70 exerts a potent immunosuppressive action on macrophages in the tumor microenvironment, the identification of this novel immunosuppressive pathway suggests innovative therapeutic possibilities. Hsp70 has been described to bind phospholipids (PLs) and this property has the potential of developing reagents that may therefore interfere with Hsp70 interaction with TLR2 and thereby inhibit macrophage differentiation. NVs derived from a panel of PLs differing in saturation and head groups were assessed for their ability to block THP-1 differentiation induced by cancer cell CMs.

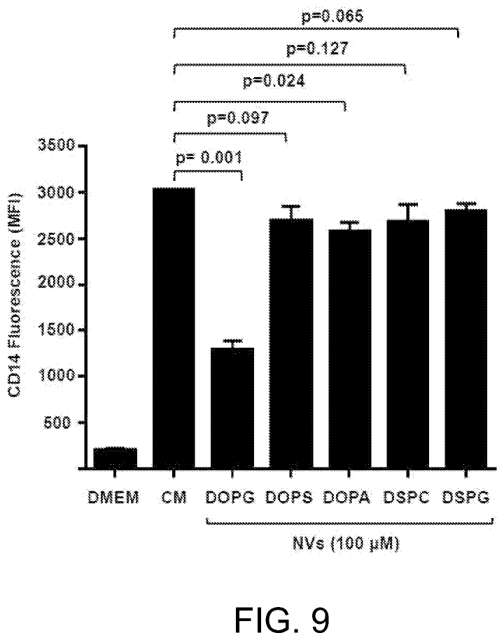

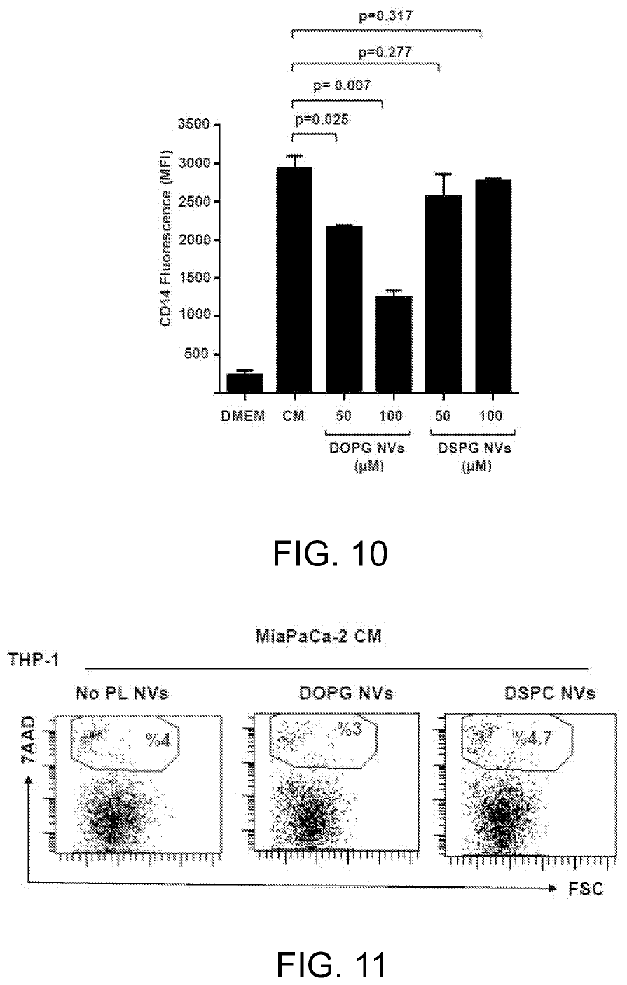

Interestingly, incubation of MiaPaCa-2 CM-treated THP-1 cells with DOPG NVs led to substantial inhibition in a dose-dependent manner of CD14 expression on THP-1 cells (FIG. 9 and FIG. 10). In comparison, there were mild inhibitions by dioleoylphosphatidic acid (DOPA) NVs, dioleoylphosphatidylserine (DOPS) NVs, distearoylphosphatidylcholine (DSPC) NVs, or distearoylphosphoglycerol (DSPG) NVs (FIGS. 9 and 10). The observed effect of DOPG NVs on THP-1 differentiation was unlikely due to toxicity, as evident by propidium iodide (PI) staining of THP-1 cells treated with DOPG NVs, which is comparable to PT staining THP-1 cells without NV treatment or DSPC NV treatment (FIG. 11). The lack of cytotoxic effect of the NVs was confirmed by treating MiaPaCa-2 cells with DOPG, DOPS, DOPA, DSPC, and DSPG NVs (100 μM) and staining with propidium iodide. Results showed the tested PG NVs did not exert a cytotoxic effect on MiaPaCa-2 cells (FIG. 16).

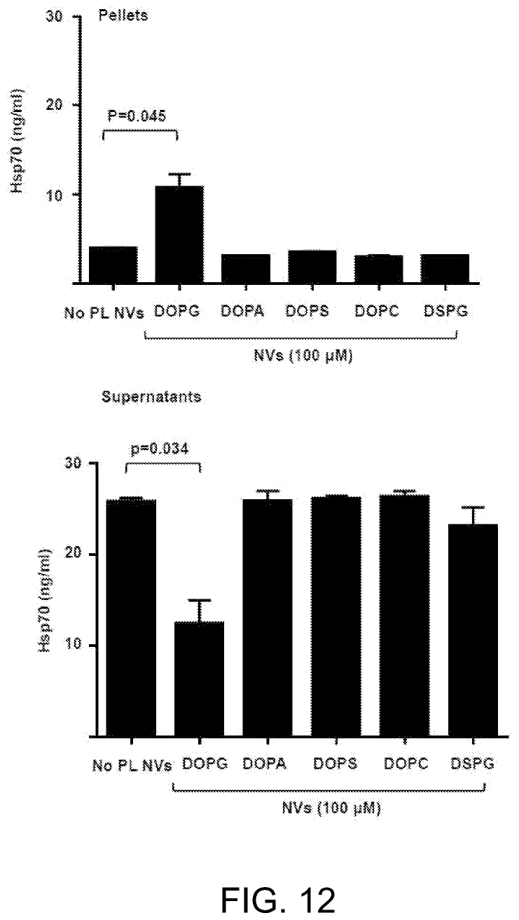

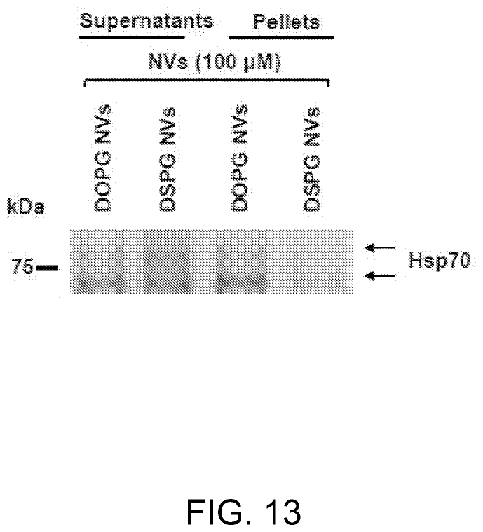

Given the observed inhibitory effect of DOPG NVs on CM-induced THP-1 differentiation, DOPG NVs were next assessed for binding to cancer-secreted Hsp70. After incubation of Gli36 CM with PL NVs, ultracentrifugation was applied to separate NV-bound-Hsp70 in the pellet from its free form in the supernatant. ELISA assays demonstrated that DOPG NVs strongly bound to Hsp70 compared to the other PL NVs as indicated by significant increase of Hsp70 in the pellet (FIG. 12, top panel) and its reduction in the supernatant (FIG. 12, bottom panel). The binding of DOPG NVs to Hsp70 was further confirmed with western blot analyses (FIG. 13). These results support the conclusion that DOPG NVs sequester cancer-secreted Hsp70 and inhibit macrophage differentiation.

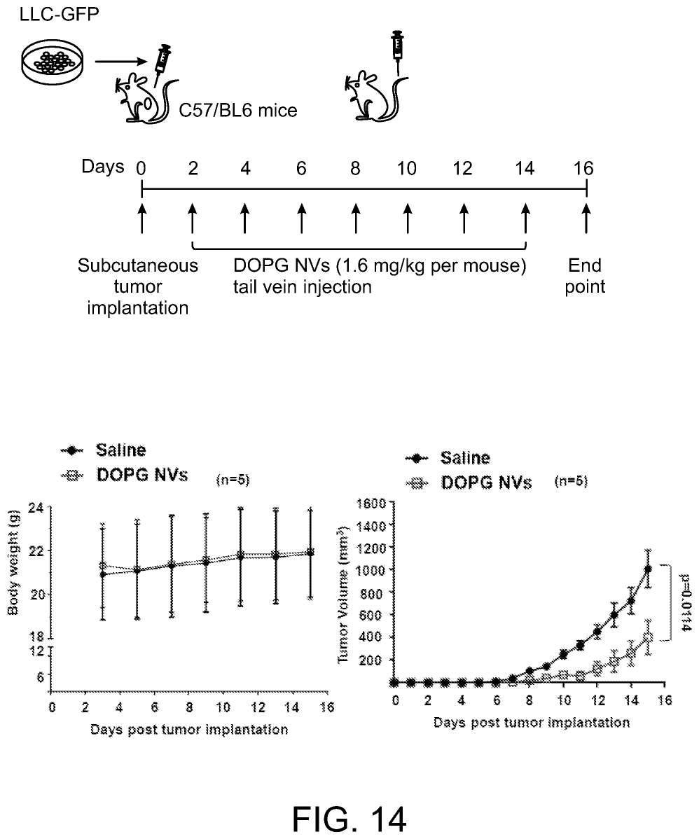

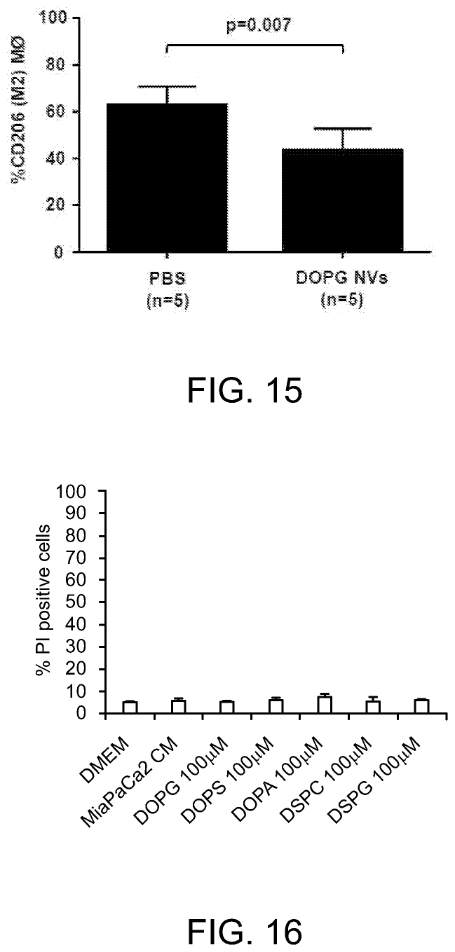

Next, the effect of DOPG NVs on tumor growth and M2 polarization in vivo was assessed. Intravenously administered DOPG NVs did not alter mice body weight, but substantially reduced tumor growth compared to sham mice (FIG. 14). Importantly, the reduction in tumor growth by DOPG NVs was associated with significant reduction in the intra-tumor M2 polarized macrophages (FIG. 15).

Aspects of the present disclosure can be described with reference to the following numbered clauses, with preferred features laid out in dependent clauses.
1. A method of treating cancer in a subject in need thereof, the method comprising administering to the subject a therapeutic amount of a composition comprising phosphatidylglycerol nanovesicles (NVs).
2. The method according to clause 1, wherein the phosphatidylglycerol has an area per lipid greater than about 70 A2.
3. The method according to any of the preceding clauses, wherein the phosphatidylglycerol NVs sequester cancer-secreted heat shock protein 70 (Hsp70).
4. The method according to any of the preceding clauses, wherein the composition does not comprise a second therapeutic agent.
5. The method according to any of the preceding clauses, wherein the phosphatidylglycerol NVs are not conjugated to a second therapeutic agent and/or do not encapsulate a second therapeutic agent.
6. The method according to any of the preceding clauses, wherein the composition is administered by injection or infusion.
7. The method according to any of the preceding clauses, wherein the cancer is selected from the group consisting of pancreatic ductal adenocarcinoma, glioblastoma, colorectal cancer, prostate cancer, hepatocellular carcinoma, lung cancer, skin cancer, cervical cancer, ovarian cancer, endometrial cancer, myeloma, acute myeloid leukemia, chronic lymphocytic leukemia, and diffuse large-B cell lymphoma.
8. The method according to any of the preceding clauses, wherein the composition consists essentially of phosphatidylglycerol NVs.
9. The method according to any of the preceding clauses, wherein the phosphatidylglycerol is selected from the group consisting of:
1,2-dioleoyl-sn-glycero-3-phosphoglycerol (DOPG);
1,2-dioleoyl-sn-glycero-3-phospho-(1′-rac-glycerol);
1,2-dielaidoyl-sn-glycero-3-phospho-(1′-rac-glycerol);
1,2-dilinoleoyl-sn-glycero-3-phospho-(1′-rac-glycerol);
1,2-dilinolenoyl-sn-glycero-3-phospho-(1′-rac-glycerol);
1,2-diarachidonoyl-sn-glycero-3-[phospho-rac-(1-glycerol)];
1,2-didocosahexaenoyl-sn-glycero-3-[phospho-rac-(1-glycerol)];
1-palmitoyl-2-oleoyl-sn-glycero-3-phospho-(1′-rac-glycerol);
1-palmitoyl-2-linoleoyl-sn-glycero-3-phospho-(1′-rac-glycerol);
1-stearoyl-2-oleoyl-sn-glycero-3-phospho-(1′-rac-glycerol);
1-stearoyl-2-linoleoyl-sn-glycero-3-phospho-(1′-rac-glycerol);
1-(10Z-heptadecenoyl)-sn-glycero-3-phospho-(1′-rac-glycerol); and
1-oleoyl-2-hydroxy-sn-glycero-3-phospho-(1′-rac-glycerol),
and combinations thereof.
   10. A method of inhibiting M2 macrophage polarization in a tumor microenvironment, the method comprising administering to the tumor microenvironment an effective amount of a composition comprising phosphatidylglycerol nanovesicles (NVs).
   11. The method according to clause 10, wherein the phosphatidylglycerol is unsaturated and has an area per lipid greater than about 70 A2.
   12. The method according to clause 10 or clause 11, wherein the method is in vitro or in vivo.
   13. The method according to any of clauses 10-12, wherein the phosphatidylglycerol NVs sequester cancer-secreted heat shock protein 70 (Hsp70).
   14. The method according to any of clauses 10-13, wherein the sequestering reduces tumor volume and/or inhibits tumor growth.
   15. The method according to any of clauses 10-14, wherein the composition does not comprise a second therapeutic agent.
   16. The method according to any of clauses 10-15, wherein the phosphatidylglycerol NVs are not conjugated to a second therapeutic agent and/or do not encapsulate a second therapeutic agent.
   17. The method according to any of clauses 10-16, wherein the composition consists essentially of phosphatidylglycerol NVs.
   18. The method according to any of clauses 10-17, wherein the tumor is selected from the group consisting of pancreatic ductal adenocarcinoma, glioblastoma, colorectal cancer, prostate cancer, hepatocellular carcinoma, lung cancer, skin cancer, cervical cancer, ovarian cancer, endometrial cancer, myeloma, acute myeloid leukemia, chronic lymphocytic leukemia, and diffuse large-B cell lymphoma.
   19. The method according to any of clauses 10-18, wherein the phosphatidylglycerol is selected from the group consisting of:
1,2-dioleoyl-sn-glycero-3-phosphoglycerol (DOPG);
1,2-dioleoyl-sn-glycero-3-phospho-(1′-rac-glycerol);
1,2-dielaidoyl-sn-glycero-3-phospho-(1′-rac-glycerol);
1,2-dilinoleoyl-sn-glycero-3-phospho-(1′-rac-glycerol);
1,2-dilinolenoyl-sn-glycero-3-phospho-(1′-rac-glycerol);
1,2-diarachidonoyl-sn-glycero-3-[phospho-rac-(1-glycerol)];
1,2-didocosahexaenoyl-sn-glycero-3-[phospho-rac-(1-glycerol)];
1-palmitoyl-2-oleoyl-sn-glycero-3-phospho-(1′-rac-glycerol);
1-palmitoyl-2-linoleoyl-sn-glycero-3-phospho-(1′-rac-glycerol);
1-stearoyl-2-oleoyl-sn-glycero-3-phospho-(1′-rac-glycerol);
1-stearoyl-2-linoleoyl-sn-glycero-3-phospho-(1′-rac-glycerol);
1-(10Z-heptadecenoyl)-sn-glycero-3-phospho-(1′-rac-glycerol); and
1-oleoyl-2-hydroxy-sn-glycero-3-phospho-(1′-rac-glycerol),
and combinations thereof.
   20. The method according to any of the preceding clauses, wherein the composition comprises phosphatidylglycerol NVs at a concentration of from about 50 μM to about 350 μM.
   21. The method according to claim any of the preceding clauses, wherein the phosphatidylglycerol is 1,2-dioleoyl-sn-glycero-3-phosphoglycerol (DOPG).
   22. The method according to any of the preceding clauses, wherein the cancer or the tumor is characterized by elevated secretion of Hsp70.
   23. A pharmaceutical composition for the treatment of cancer, comprising:
a therapeutic amount of phosphatidylglycerol nanovesicles (NVs); and
at least one pharmaceutically acceptable excipient,
wherein the phosphatidylglycerol NVs do not encapsulate a second therapeutic agent and are not conjugated to a second therapeutic agent.
   24. The pharmaceutical composition according to clause 23, wherein the phosphatidylglycerol is unsaturated and has an area per lipid (APL) greater than about 70 A2.
   25. The pharmaceutical composition according to clause 23 or clause 24, wherein the composition is formulated for injection or infusion.
   26. The pharmaceutical composition according to any of clauses 23-25, wherein the phosphatidylglycerol NVs are the sole therapeutic agent in the pharmaceutical composition.
   27. The pharmaceutical composition according to any of clauses 23-26, wherein the phosphatidylglycerol is selected from the group consisting of:
1,2-dioleoyl-sn-glycero-3-phosphoglycerol (DOPG);
1,2-dioleoyl-sn-glycero-3-phospho-(1′-rac-glycerol);
1,2-dielaidoyl-sn-glycero-3-phospho-(1′-rac-glycerol);
1,2-dilinoleoyl-sn-glycero-3-phospho-(1′-rac-glycerol);
1,2-dilinolenoyl-sn-glycero-3-phospho-(1′-rac-glycerol);
1,2-diarachidonoyl-sn-glycero-3-[phospho-rac-(1-glycerol)];
1,2-didocosahexaenoyl-sn-glycero-3-[phospho-rac-(1-glycerol)];
1-palmitoyl-2-oleoyl-sn-glycero-3-phospho-(1′-rac-glycerol);
1-palmitoyl-2-linoleoyl-sn-glycero-3-phospho-(1′-rac-glycerol);
1-stearoyl-2-oleoyl-sn-glycero-3-phospho-(1′-rac-glycerol);
1-stearoyl-2-linoleoyl-sn-glycero-3-phospho-(1′-rac-glycerol);
1-(10Z-heptadecenoyl)-sn-glycero-3-phospho-(1′-rac-glycerol); and
1-oleoyl-2-hydroxy-sn-glycero-3-phospho-(1′-rac-glycerol),
and combinations thereof.
   28. The pharmaceutical composition according to any of clauses 23-27, wherein the phosphatidylglycerol is 1,2-dioleoyl-sn-glycero-3-phosphoglycerol (DOPG).
   29. The pharmaceutical composition according to any of clauses 23-28, wherein the composition comprises phosphatidylglycerol NVs at a concentration of from about 50 μM to about 350 μM.
   30. A composition comprising phosphatidylglycerol nanovesicles (NVs) for use in a method of treating cancer, the method comprising administering the composition to the subject.
   31. The composition for use according to clause 30, wherein the cancer is characterized by elevated secretion of Hsp70.
   32. The composition for use according to any of clauses 30-31, wherein the phosphatidylglycerol has an area per lipid greater than about 70 A2.
   33. The composition for use according to any of clauses 30-32, wherein the phosphatidylglycerol NVs sequester cancer-secreted heat shock protein 70 (Hsp70).
   34. The composition for use according to any of clauses 30-33, wherein the composition does not comprise a second therapeutic agent.
   35. The composition for use according to any of clauses 30-34, wherein the phosphatidylglycerol NVs are not conjugated to a second therapeutic agent and/or do not encapsulate a second therapeutic agent.
   36. The composition for use according to any of clauses 30-35, wherein the composition is administered by injection or infusion.
   37. The composition for use according to any of clauses 30-36, wherein the cancer is selected from the group consisting of pancreatic ductal adenocarcinoma, glioblastoma, colorectal cancer, prostate cancer, hepatocellular carcinoma, lung cancer, skin cancer, cervical cancer, ovarian cancer, endometrial cancer, myeloma, acute myeloid leukemia, chronic lymphocytic leukemia, and diffuse large-B cell lymphoma.
   38. The composition for use according to any of clauses 30-37, wherein the composition consists essentially of phosphatidylglycerol NVs.
   39. The composition for use according to any of clauses 30-38, wherein the phosphatidylglycerol is selected from the group consisting of:
1,2-dioleoyl-sn-glycero-3-phosphoglycerol (DOPG);
1,2-dioleoyl-sn-glycero-3-phospho-(1′-rac-glycerol);
1,2-dielaidoyl-sn-glycero-3-phospho-(1′-rac-glycerol);
1,2-dilinoleoyl-sn-glycero-3-phospho-(1′-rac-glycerol);
1,2-dilinolenoyl-sn-glycero-3-phospho-(1′-rac-glycerol);
1,2-diarachidonoyl-sn-glycero-3-[phospho-rac-(1-glycerol)];
1,2-didocosahexaenoyl-sn-glycero-3-[phospho-rac-(1-glycerol)];
1-palmitoyl-2-oleoyl-sn-glycero-3-phospho-(1′-rac-glycerol);
1-palmitoyl-2-linoleoyl-sn-glycero-3-phospho-(1′-rac-glycerol);
1-stearoyl-2-oleoyl-sn-glycero-3-phospho-(1′-rac-glycerol);
1-stearoyl-2-linoleoyl-sn-glycero-3-phospho-(1′-rac-glycerol);
1-(10Z-heptadecenoyl)-sn-glycero-3-phospho-(1′-rac-glycerol); and
1-oleoyl-2-hydroxy-sn-glycero-3-phospho-(1′-rac-glycerol),
and combinations thereof.
   40. The composition for use according to any of clauses 30-39, wherein the phosphatidylglycerol is DOPG.
   41. The composition for use according to any of clauses 30-40, wherein the composition comprises phosphatidylglycerol NVs at a concentration of from about 50 μM to about 350 μM.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A method of treating cancer in a subject in need thereof, the method comprising administering to the subject a therapeutic amount of a composition comprising phosphatidylglycerol nanovesicles (NVs).

2. The method according to claim 1, wherein the phosphatidylglycerol has an area per lipid greater than about 70 Å².

3. The method according to claim 1, wherein the phosphatidylglycerol NVs sequester cancer-secreted heat shock protein 70 (Hsp70).

4. The method according to claim 1, wherein the composition does not comprise a second therapeutic agent.

5. The method according to claim 1, wherein the phosphatidylglycerol NVs are not conjugated to a second therapeutic agent and/or do not encapsulate a second therapeutic agent.

6. The method according to claim 1, wherein the composition is administered by injection or infusion.

7. The method according to claim 1, wherein the cancer is selected from the group consisting of pancreatic ductal adenocarcinoma, glioblastoma, colorectal cancer, prostate cancer, hepatocellular carcinoma, lung cancer, skin cancer, cervical cancer, ovarian cancer, endometrial cancer, myeloma, acute myeloid leukemia, chronic lymphocytic leukemia, and diffuse large-B cell lymphoma.

8. The method according to claim 1, wherein the composition consists essentially of phosphatidylglycerol NVs.

9. The method according to claim 1, wherein the phosphatidylglycerol is selected from the group consisting of:
1,2-dioleoyl-sn-glycero-3-phosphoglycerol (DOPG);
1,2-dioleoyl-sn-glycero-3-phospho-(1′-rac-glycerol);
1,2-dielaidoyl-sn-glycero-3-phospho-(1′-rac-glycerol);
1,2-dilinoleoyl-sn-glycero-3-phospho-(1′-rac-glycerol);
1,2-dilinolenoyl-sn-glycero-3-phospho-(1′-rac-glycerol);
1,2-diarachidonoyl-sn-glycero-3-[phospho-rac-(1-glycerol)];
1,2-didocosahexaenoyl-sn-glycero-3-[phospho-rac-(1-glycerol)];
1-palmitoyl-2-oleoyl-sn-glycero-3-phospho-(1′-rac-glycerol);
1-palmitoyl-2-linoleoyl-sn-glycero-3-phospho-(1′-rac-glycerol);
1-stearoyl-2-oleoyl-sn-glycero-3-phospho-(1′-rac-glycerol);
1-stearoyl-2-linoleoyl-sn-glycero-3-phospho-(1′-rac-glycerol);
1-(10Z-heptadecenoyl)-sn-glycero-3-phospho-(1′-rac-glycerol); and
1-oleoyl-2-hydroxy-sn-glycero-3-phospho-(1′-rac-glycerol),
and combinations thereof.

10-19. (canceled)

20. The method according to claim 1, wherein the composition comprises phosphatidylglycerol NVs at a concentration of from about 50 μM to about 350 μM.

21. The method according to claim 1, wherein the phosphatidylglycerol is 1,2-dioleoyl-sn-glycero-3-phosphoglycerol (DOPG).

22. The method according to claim 1, wherein the cancer or the tumor is characterized by elevated secretion of Hsp70.

23. A pharmaceutical composition for the treatment of cancer, comprising:
a therapeutic amount of phosphatidylglycerol nanovesicles (NVs); and
at least one pharmaceutically acceptable excipient,
wherein the phosphatidylglycerol NVs do not encapsulate a second therapeutic agent and are not conjugated to a second therapeutic agent.

24. The pharmaceutical composition according to claim 23, wherein the phosphatidylglycerol is unsaturated and has an area per lipid (APL) greater than about 70 Å².

25. The pharmaceutical composition according to claim 23, wherein the composition is formulated for injection or infusion.

26. The pharmaceutical composition according to claim 23, wherein the phosphatidylglycerol NVs are the sole therapeutic agent in the pharmaceutical composition.

27. The pharmaceutical composition according to claim 23, wherein the phosphatidylglycerol is selected from the group consisting of:
1,2-dioleoyl-sn-glycero-3-phosphoglycerol (DOPG);
1,2-dioleoyl-sn-glycero-3-phospho-(1′-rac-glycerol);
1,2-dielaidoyl-sn-glycero-3-phospho-(1′-rac-glycerol);
1,2-dilinoleoyl-sn-glycero-3-phospho-(1′-rac-glycerol);
1,2-dilinolenoyl-sn-glycero-3-phospho-(1′-rac-glycerol);
1,2-diarachidonoyl-sn-glycero-3-[phospho-rac-(1-glycerol)];
1,2-didocosahexaenoyl-sn-glycero-3-[phospho-rac-(1-glycerol)];
1-palmitoyl-2-oleoyl-sn-glycero-3-phospho-(1′-rac-glycerol);
1-palmitoyl-2-linoleoyl-sn-glycero-3-phospho-(1′-rac-glycerol);
1-stearoyl-2-oleoyl-sn-glycero-3-phospho-(1′-rac-glycerol);
1-stearoyl-2-linoleoyl-sn-glycero-3-phospho-(1′-rac-glycerol);
1-(10Z-heptadecenoyl)-sn-glycero-3-phospho-(1′-rac-glycerol); and
1-oleoyl-2-hydroxy-sn-glycero-3-phospho-(1′-rac-glycerol),
and combinations thereof.

28. The pharmaceutical composition according to claim 27, wherein the phosphatidylglycerol is 1,2-dioleoyl-sn-glycero-3-phosphoglycerol (DOPG).

29. The pharmaceutical composition according to claim 23, wherein the composition comprises phosphatidylglycerol NVs at a concentration of from about 50 μM to about 350 μM.

30-41. (canceled)

42. A method of inhibiting M2 macrophage polarization in a tumor microenvironment, the method comprising administering to the tumor microenvironment the pharmaceutical composition according to claim 23.
